(19) United States
(12) Patent Application Publication
(10) Pub. No.: US 2010/0130880 A1
(43) Pub. Date: May 27, 2010

(54) APPARATUS AND METHODS FOR MONITORING BLOOD FLOW IN THE PROSTRATE GLAND

(75) Inventor: Luya Li, Coquitlam (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: URODYNAMIX TECHNOLOGIES LTD., Vancouver, BC (CA)

(21) Appl. No.: 12/595,338

(22) PCT Filed: Apr. 16, 2008
(86) PCT No.: PCT/CA2008/000698

§ 371 Date: Oct. 9, 2009

Related U.S. Application Data
(60) Provisional application No. 60/912,125, filed on Apr. 16, 2007.
(60) Provisional application No. 61/028,870, filed on Feb. 14, 2008.

(51) Int. Cl. A61B 5/0265

(52) U.S. Cl. 600/504

(57) ABSTRACT

A system for detecting blood flow in the prostate comprises a blood flow sensor disposed on a catheter that can be inserted into a subject's urethra so that the blood flow sensor is located to detect blood flow in the subject's prostate gland. The sensor may be a sensor of a near infrared spectroscopy (NIRS) system configured to detect in the prostate one or more biocompounds indicative of blood flow. An output of the sensor may provide an input to a controller for a heater disposed to heat tissues of the prostate. Some embodiments comprise one or more additional sensors for detecting blood flow and/or temperature of a portion of the subject's rectal wall adjacent to the prostate gland. In such embodiments, outputs from the additional sensors may provide additional inputs to the controller.

### CROSS-REFERENCE TO RELATED APPLICATIONS

This application claims priority from U.S. patent application No. 61/028,870 filed 14 Feb. 2008 and U.S. patent application No. 60/912,125 filed 16 Apr. 2007. For purposes of the United States of America, this application claims the benefit under 35 U.S.C. §119 of U.S. patent application No. 61/028,870 filed 14 Feb. 2008 and U.S. patent application No. 60/912,125 filed 16 Apr. 2007, both of which are hereby incorporated by reference herein.

### TECHNICAL FIELD

This invention relates to monitoring blood flow in the prostate gland. Embodiments of the invention provide methods and systems for treating tumors or other tissues within the prostate gland.

### BACKGROUND

Prostate cancer, benign prostatic hyperplasia and other conditions can cause the prostate to become enlarged. These conditions can result in urinary blockage and other adverse effects on health. These conditions are sometimes treated by selectively heating tissues of the prostate gland. Some systems for heating the prostate gland and tissues use a temperature sensor inserted in the subject's rectum to measure the amount of heat being delivered to the prostate gland. Since the rectum passes close to the prostate gland, the temperature measured in the rectum can be useful in controlling the application of heat to raise the temperature of the prostate gland.

There is a need for improved methods and apparatus for heating the prostate gland which provide better control over the temperatures achieved in the prostate gland.

Near Infrared Spectroscopy (“NIRS”) is a technique which involves emitting near infrared (“NIR”) light and receiving the NIR light after it has passed through a tissue or other medium of interest. NIRS can be applied to study and monitor biochemical compounds in the body. Emitted NIR light penetrates skin and other tissues and some of it is absorbed by biochemical compounds which have an absorption spectrum in the NIR region. NIR light which is not absorbed is scattered. Each biochemical compound has a different absorption spectrum. It is possible to estimate the concentration of biochemical compounds in the tissues by measuring characteristics of NIR light that has been detected after it has passed through the tissues. The use of NIRS to measure changes in concentrations of various compounds in living tissues by monitoring appropriate wavelengths is understood by those of skill in the art.

### SUMMARY

This invention provides a range of methods and apparatus that can be used together in various combinations, can be used individually or can be used in combination with other methods and apparatus.

Various non-limiting example aspects of the invention and features of example embodiments of the invention are described below.

### BRIEF DESCRIPTION OF THE DRAWINGS

The accompanying drawings illustrate non-limiting embodiments of the invention.

FIG. 1 is a cross section through a subject in which apparatus according to the invention is deployed.

FIG. 2 is a cross section through a portion of a catheter in a example embodiment of the invention.

FIG. 3 is a cross section through a catheter in an alternative embodiment of the invention.

FIG. 4 is a cross section through a catheter according to a further alternative embodiment of the invention.

FIG. 5 is a cross section through a subject in which apparatus according to another embodiment of the invention is deployed.

FIG. 6 is an enlarged schematic view of a rectal probe of the apparatus of FIG. 5 and associated control systems.

FIG. 7 is a schematic plot illustrating gross features of the variation with time of blood flow in a portion of the rectal wall adjacent to a subject's prostate during heat treatment of the prostate.

### DETAILED DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

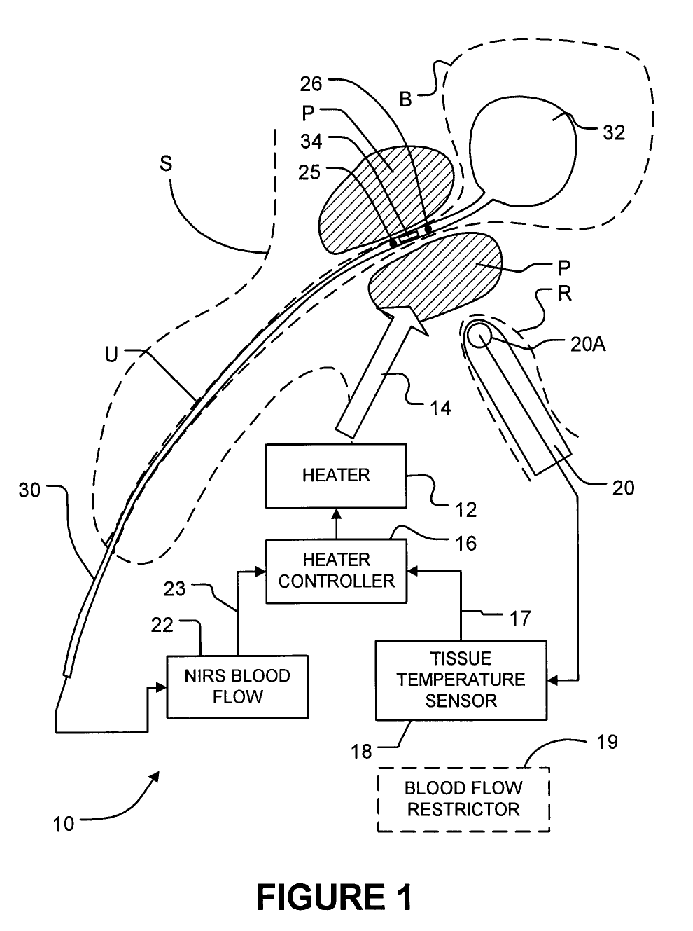

FIG. 1 is a partially schematic view of apparatus according to one embodiment of the invention deployed in a subject S. Subject S has a bladder B. The subject's urethra U extends out of bladder B. Urethra U passes through the prostate gland P. If prostate gland P becomes enlarged or swollen then blockage of urethra U can occur. This can result in significant complications and discomfort.

Apparatus 10 comprises a heater 12 that directs energy 14 toward the subject's prostate gland P. Heater 12 may heat the tissues of the prostate using any suitable modality. For example, heater 12 may comprise one or more of:
a microwave heater that emits microwave energy that is absorbed in the prostate;
an emitter of light (either visible or invisible light that is absorbed in the prostate and converted to heat);
a radio frequency emitter that emits radio frequency radiation that is absorbed in the prostate;
a source of ultrasound energy that is absorbed in the prostate;
or the like.

The output of heater 12 is controlled by a heater controller 16. Heater controller 16 takes as an input a tissue temperature signal 17 from a tissue temperature sensor 18. In the illustrated embodiment, tissue temperature sensor 18 comprises a probe 20 that is inserted into the rectum R of the subject. Probe 20 senses the temperature at a point 20A which is adjacent to the subject's prostate when probe 20 is inserted into the subject's rectum R. Temperature sensor 18 and its associated probe 20 are desirable but optional.

Heater controller 16 may provide closed-loop control of energy 14 based on feedback from temperature sensor 18 and/or other sensors to achieve a desired temperature within the tissues of prostate P.

Apparatus 10 also includes a near infrared spectrometer system (NIRS system) 22 that monitors blood flow in the subject's prostate. Near infrared spectrometry is a known technique that can be used to monitor for changes in the concentrations of various bio-compounds in living tissues. For example, NIRS can be used to monitor the concentrations of one or more of:
oxygenated hemoglobin (HbO₂);
non-oxygenated hemoglobin (Hb); and
total hemoglobin (HbTot);
   in the prostate or in tissues surrounding the prostate. These concentrations vary with the blood volume which, in turn, varies with blood flow. Levels of cytochrome and/or myoglobin may also be monitored to provide additional information regarding the condition of the prostate.

NIRS involves directing near infrared light from a light source into tissues of interest and detecting the infrared light after it has passed through the tissues of interest. In the illustrated embodiment, a NIRS light source 25 and light detector 26 are provided on a catheter 30 that can be inserted through the subject's urethra U. An anchoring structure is provided to retain catheter 30 in the subject's urethra. In the illustrated embodiment, the anchoring structure comprises a balloon 32 on the distal end of catheter 30. Balloon 32 can be inflated after the distal end of catheter 30 has passed into the subject's bladder B. Balloon 32 may, for example, be inflated by pumping a fluid into balloon 32 through catheter 30. Balloon 32 retains catheter 30 in the subject's urethra.

Catheters for insertion into the urethra are known. The details of construction and operation of catheter 30 are not described herein since such details can readily be developed by those skilled in the art, for example by reference to commercially-available catheters.

Light source 25 and light detector 26 are spaced apart from balloon 32 by a distance which is suitable to locate source 25 and light detector 26 in the portion of the subject's urethra U that passes through the subject's prostate. Light emitted by light source 25 is back-scattered by tissues in the subject's prostate P and picked up at light detector 26. An amount of one or more bio compounds related to blood flow for example, oxygenated hemoglobin (HbO₂); non-oxygenated hemoglobin (Hb); and/or total hemoglobin (HbTot) may be determined by processing the signal from light detector 26. The result is that NIRS spectrometer 22 generates a signal 23 indicative of blood flow in the subject's prostate.

Blood flow in the subject's prostate is important because flowing blood can carry away heat from the subject's prostate. Heater controller 16 may be programmed to increase the output of heater 12 as blood flow increases (e.g. in response to a measure of blood flow determined by NIRS system 22), so as to suitably increase the amount of heat deposited in tissues of the subject's prostate P to compensate at least partially for heat that is carried away by flowing blood, and thereby raise those tissues to a desired temperature.

In some embodiments, heater 12 delivers energy 14 to the subject's prostate P by way of catheter 30. For example, energy 14 could be microwave energy that is delivered from a microwave antenna 34 supported in or on catheter 30. In other embodiments, energy 14 is delivered from outside of the subject. Any suitable mechanism for heating the tissues of the prostate may be used.

There are a wide range of ways in which each of NIRS light source 25 and light detector 26 may be provided in a catheter 30. These can be applied in any combination. Some such combinations are disclosed in the illustrated embodiments.

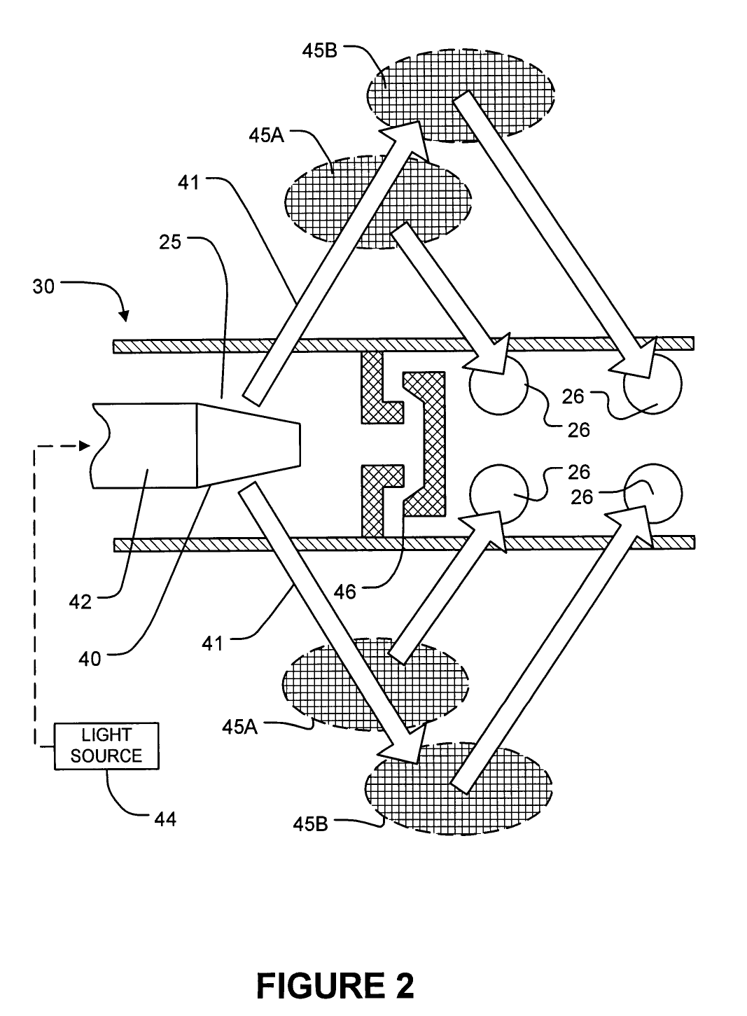

FIG. 2 shows a cross section through one possible configuration of catheter 30. In the illustrated embodiment, light source 25 comprises a diffuser 40 on the end of an optical fibre 42 that carries optical radiation from a light source 44. The light propagates along optical fibre 42 from light source 44 to diffuser 40. At diffuser 40 light 41 is directed out into the tissues surrounding catheter 30.

As an alternative to carrying light along catheter 30 in an optical fibre 42, NIRS light sources such as suitable light-emitting diodes (LEDs) could be provided in catheter 30 and supplied with electrical power by way of wires or other electrical conductors extending along catheter 30.

Light detectors 26 comprise photo transistors, photo-diodes, or other detectors sensitive to light at the wavelengths emitted by light source 25. In the illustrated embodiment, there is a plurality of light sensors 26 that sense light incident from different directions and which also include light sensors 26 that are spaced apart from light source 25 by different distances. The depth within the tissues at which the concentration(s) of the monitored bio-compound(s) is measured depends in part on the separation between light source 25 and a light detector 26. By providing light detectors 26 that are separated from light source 25 by different distances one can determine the blood flow at different depths in the prostate. In FIG. 2, the amount of haemoglobin (which varies with blood flow) is detected in regions 45A, which are closer to catheter 30 and also in regions 45B, which are farther from catheter 30. In some embodiments, the NIRS light sources and light detectors include pairs of light sources and light detectors that are spaced apart along catheter 30 by distances in the range of about ½ cm to 2^{1/2} cm.

A light barricade 46 is disposed between light source 25 and light detectors 26 to prevent light from passing directly from light source 25 to light detectors 26. In the illustrated embodiment, light barricade 46 has a labyrinth construction to permit the flow of a fluid to inflate balloon 32.

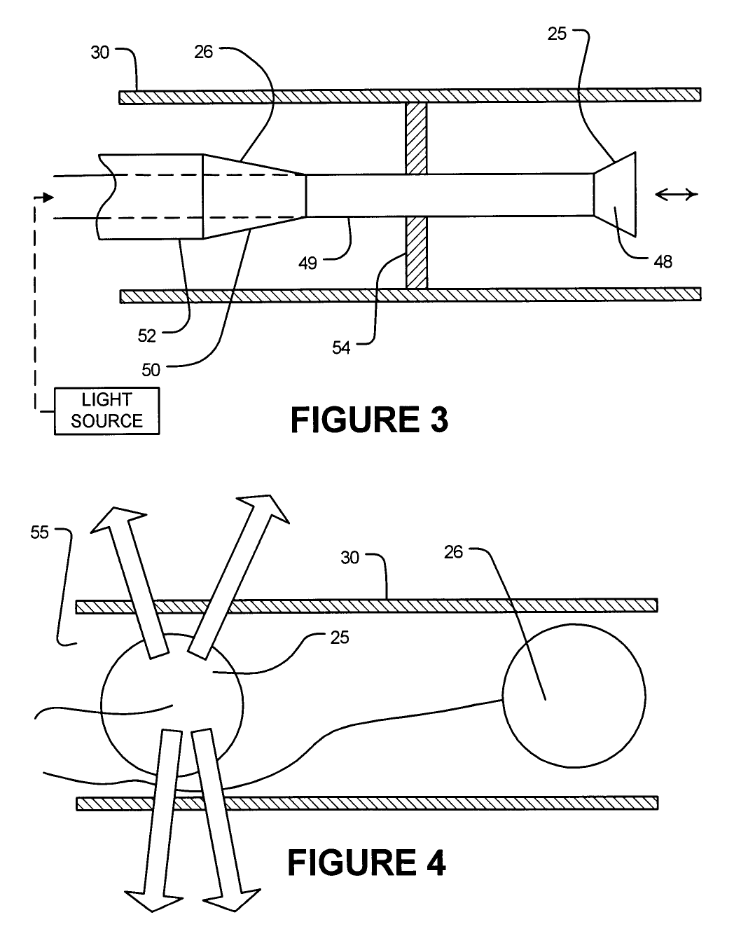

FIG. 3 shows an alternative embodiment of a portion of catheter 30 in which light source 25 comprises a diffuser 48 which releases light propagating in an optical fibre 49. Light detector 26 comprises a light collector 50 disposed on the end of an optical fibre 52. In the illustrated embodiment, optical fibre 52 is coaxial with optical fibre 49. In the illustrated embodiment, the separation between light source 25 and light detector 26 can be varied by sliding optical fibre 52 within optical fibre 49. A light barrier 54 prevents light from light source 25 from directly reaching light detector 26.

FIG. 4 shows an alternative embodiment in which light source 25 and light detector 26 are both discrete devices located in a lumen 55 of catheter 30. At least in its portions adjacent to light emitter 25 and light detector 26, the walls of catheter 30 are transparent to the radiation emitted by light source 25. Light emitter 25 and light detector 26 may be fixed in catheter 30 or one or both of light emitter 25 and light detector 26 may be movable.

As can be appreciated, the construction of the NIRS light source and receiver may be varied in many ways. In some embodiments, one fixed light source and one fixed light receiver may be provided. The light source may emit light that is directed in a particular direction or may emit light so that it radiates in all directions from catheter 30. In some embodiments, the separation between the light source and detector can be varied. In some embodiments there are a number of pairs of light sources and detectors (for example, one light source and a plurality of different light detectors) in such embodiments the light detectors may be spaced apart by different distances from the light sources so as to sample the concentrations of the bio-compounds being monitored at various depths in the tissue of the prostate gland.

In embodiments where there are a plurality of different light receivers 26, the light receivers 26 may receive light incident on catheter 30 from different directions, thereby making it possible to sense the concentrations of monitored bio-compounds in tissues located on different sides of catheter 30.

In some embodiments, heater controller 16 switches heater 12 on and off (or modulates the output of energy 14) in response to the detection of blood flow by NIRS system 22. Treatment efficacy can be improved by applying heat to tissues of the prostate during periods in which blood flow is below a threshold value and not applying heat during periods wherein the blood flow exceeds the threshold value.

In some embodiments, apparatus 10 includes a mechanism 19 (see FIG. 1) for controlling blood flow to the prostate. The mechanism may, for example, comprise a mechanism that clamps off or restricts blood flow in the arteries that supply the prostate. In such embodiments, the mechanism may operate periodically to reduce blood flow in the prostate. The heater controller may cause the heater to operate during such periods of reduced blood flow.

In some embodiments, a NIRS light source and receiver are provided on a rectal probe. In such embodiments, the NIRS light source and receiver on the rectal probe may provide an output indicative of blood flow in a portion of the rectal wall close to a subject's prostate. Changes in the blood flow in the rectal wall can signal the onset of damage to tissues of the rectal wall. Such damage could be caused, for example, by overly-long and/or overly-intense treatment by a heater 12 as described above.

In some embodiments, a controller for a heater receives a rectal-wall-blood-flow signal indicative of blood flow in the rectal wall and causes the heater to be reduced in intensity and/or shut down when the rectal-wall-blood-flow signal satisfies a criterion. The criterion may be selected to cause the heating to be reduced or stopped prior to the rectal wall undergoing significant damage.

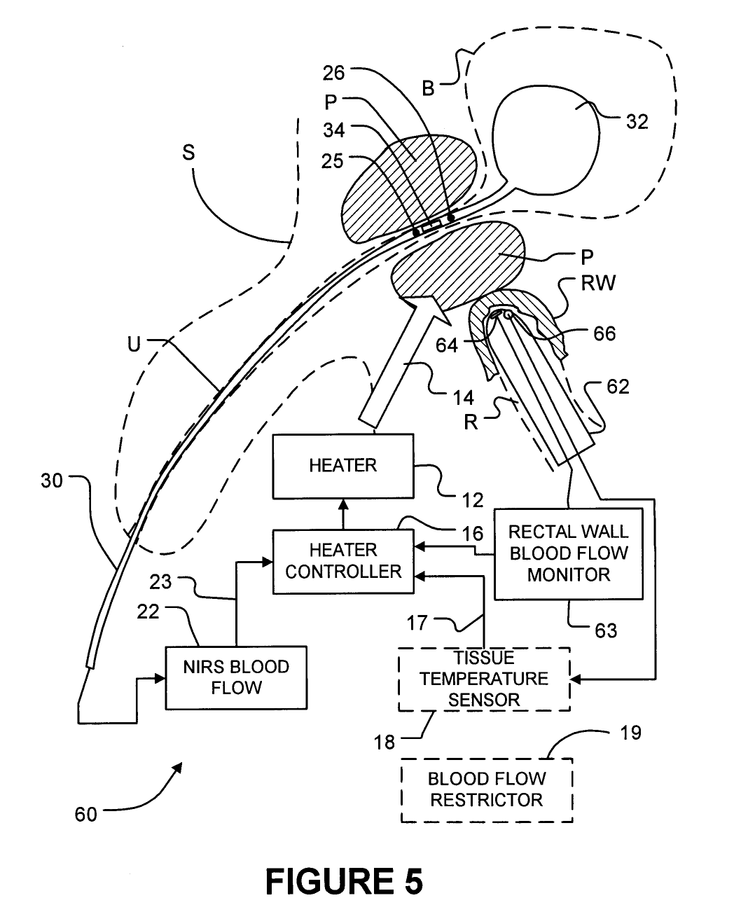

FIG. 5 shows an example embodiment wherein apparatus 60 is deployed in a subject S. FIG. 5 uses the same reference characters as FIG. 1 to identify parts of the subject. FIG. 5 identifies some parts of apparatus 60 that are also found in apparatus 10 with the same reference characters as used in FIG. 1. Apparatus 60 differs from apparatus 10 in that it has a probe 62 which supports a rectal-wall blood-flow sensor 64. Probe 62 optionally also supports a temperature sensor 66 (which may be like temperature sensor 20A). Heater controller 16 receives signals from rectal-wall blood-flow sensor 64 and also from temperature sensor 66, if present.

In the illustrated embodiment, a rectal wall blood flow monitor 63 receives signals from rectal-wall blood-flow sensor 64 and outputs a rectal wall blood flow signal. In cases where rectal-wall blood-flow sensor 64 generates a signal indicative of blood flow in the rectal wall that can be used directly by heater controller 16 the rectal wall blood flow monitor may not be present or may provide signal conditioning.

Rectal-wall blood-flow sensor 64 may comprise any of a variety of blood-flow sensors. For example, Rectal-wall blood-flow sensor 64 may comprise:
a NIRS sensor;
an ultrasound-based blood-flow detector;
a skin impedance detector; or,
the like.

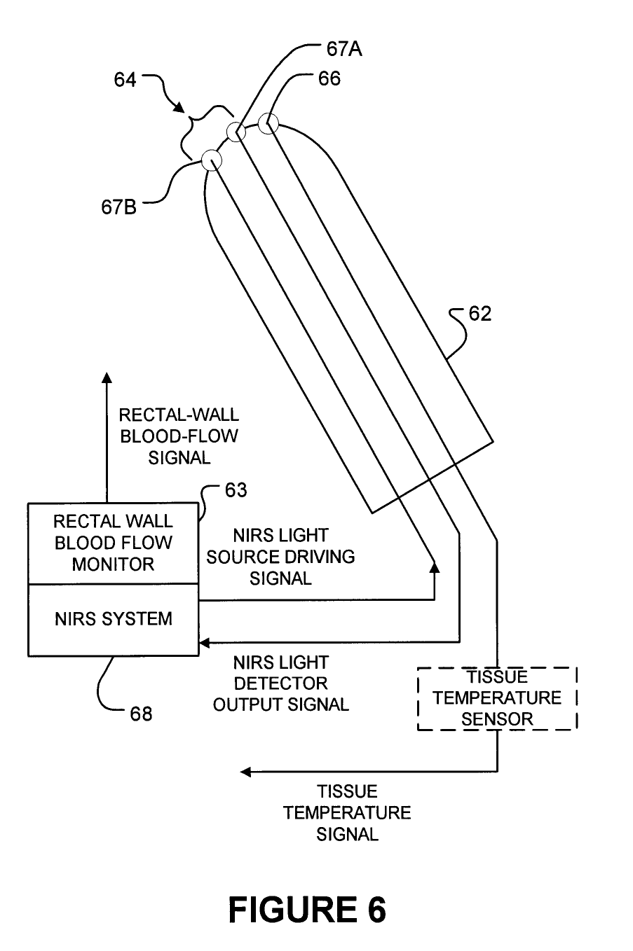

FIG. 6 shows a schematic view of a probe 62 wherein rectal-wall blood-flow sensor 64 comprises a NIRS sensor that includes a NIRS light source 67A spaced apart from a NIRS light detector 67B. Sensor 64 is on an aspect of probe 62 that can be placed against the portion of the subject's rectal wall RW that is adjacent to the prostate P.

A NIRS system 68 supplies driving signals to NIRS light source 67A and receives signals from NIRS light detector 67B. NIRS system 68 processes signals received from NIRS light source 67A to derive an output signal indicative of blood flow in the rectal wall. The output signal is supplied to heater controller 16.

In some embodiments, the NIRS system 68 generates outputs which represent concentrations in the rectal wall of one or more of:
oxygenated hemoglobin;
non-oxygenated hemoglobin;
total hemoglobin;
myoglobin.
   In some embodiments, rectal wall blood flow monitor generates the rectal-wall blood-flow signal based on one or more of these concentrations.

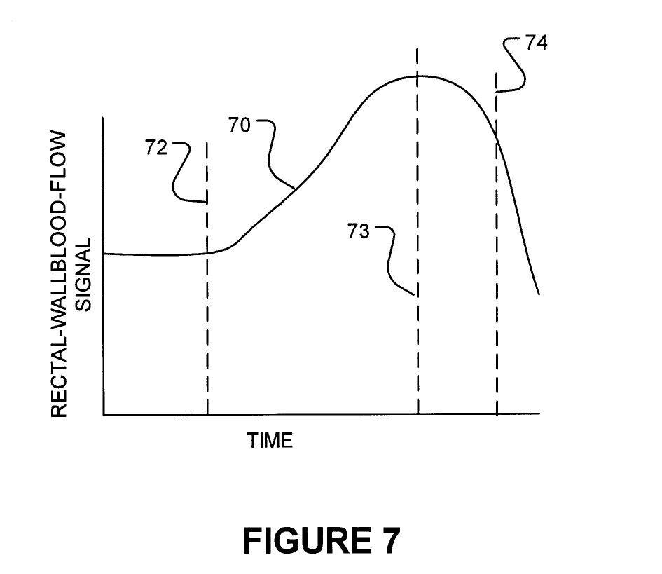

FIG. 7 shows schematically a curve 70 illustrating a variation with time of a rectal-wall-blood-flow signal indicative of blood flow in the portion of a subject's rectal wall near the subject's prostate as might be observed while the subject is receiving a treatment which involves heating the subject's prostate.

A heat treatment is initiated at time 72. As the heat begins to act on the subject's prostate and to heat surrounding tissues, blood flow in the subject's rectal wall initially increases. As the rectal wall begins to suffer damage, the blood flow peaks at 73. When the rectal wall has become seriously damaged, the blood flow in the damaged portion of the rectal wall declines precipitously. Curve 70 is schematic only and is not based on any specific clinical data.

Controller 16 may reduce the output of, or shut off, heater 12 by determining that the rectal-wall-blood-flow signal matches a criterion. The criterion may include, for example:
determining that the rectal-wall-blood-flow signal has peaked;
determining that the rectal-wall-blood-flow signal is decreasing with time at a rate exceeding a threshold;
determining that the rectal-wall-blood-flow signal is less than or equals a threshold value;
a combination thereof; or
the like.
   Where the rectal-wall-blood-flow signal is compared to a threshold, the threshold may be based on one or more of:
a value of the rectal-wall-blood-flow signal prior to or at the outset of treatment (e.g. a value of the rectal-wall-blood-flow signal at a time at or near to time 72);
a value of the rectal-wall-blood-flow signal at peak 73;
a predetermined value;
a value of the rectal-wall-blood-flow signal at a time 74 when a rate of change of the rectal-wall-blood-flow signal with time reaches a threshold amount; and
the like.

Apparatus 60 preferably operates to prevent heater 12 from operating in a way that causes lasting damage to the subject's rectal wall while allowing heater 12 to operate in a way that will provide sufficient heating to the subject's prostate P to achieve a desired treatment outcome.

In alternative embodiments, probe 60 may comprise a plurality of NIRS light sources 67A and/or a plurality of NIRS light detectors 67B. In such embodiments, the light sources and light detectors may be arranged in various ways. For example:
The NIRS light sources and detectors may include pairs of light sources and detectors that are spaced apart from one another by different distances. This permits determining the concentrations of substances such as hemoglobin, at different depths in the adjacent tissue. In general, other factors being equal, NIRS samples more deeply when the NIRS light source and detector are more widely-separated and samples at shallower volumes within adjacent tissues where the NIRS light source and detector are more closely-spaced. In some embodiments, a single NIRS light source (or set of light sources) is associated with a plurality of different NIRS light detectors that are spaced apart from the NIRS light source by different distances. In some embodiments a single NIRS light detector is associated with a plurality of different NIRS light sources that are spaced apart from the NIRS light detector by different distances. For example, the NIRS light source and NIRS light detectors may be arranged at different locations along a straight line or curve. In some embodiments a single NIRS light detector is associated with a plurality of different NIRS light sources that are spaced apart from the NIRS light detector by different distances. In some embodiments, NIRS light sources are paired with NIRS light detectors in a 1:1 relationship.
The NIRS light sources and detectors may include pairs of light sources and detectors that face in different directions from probe 62. Such embodiments may permit measurement of blood flow in a plurality of different areas of the rectal wall.
The NIRS light sources and detectors may be arranged to provide a plurality of pairs of NIRS light sources and detectors spaced apart by different amounts on each of a plurality of different aspects of probe 62 to permit measurement of blood flow at a plurality of different depths at each of a plurality of different areas of the rectal wall.

In embodiments that provide a plurality of pairs of NIRS light sources and detectors, signals from the different pairs of NIRS light sources and detectors may be applied in different ways. In some embodiments, a rectal-wall-blood-flow signal is derived from each of a plurality of NIRS output signals and the rectal-wall-blood-flow signals are each treated separately. In such embodiments, if any one of the rectal-wall-blood-flow signals indicates impending tissue damage to the rectal wall then heater controller 16 may be configured to shut off or reduce the output from heater 12. In some embodiments, a rectal-wall-blood-flow signal is obtained from a plurality of NIRS output signals. For example: NIRS output signals from differently-spaced pairs of NIRS light sources and receivers may be processed and combined to provide a rectal-wall-blood-flow signal characteristic of blood flow at both shallower and deeper parts of the rectal wall; and/or NIRS output signals from pairs of NIRS light sources and receivers facing in different directions may be combined to provide a rectal-wall-blood-flow signal representative of blood flow over a larger area of the rectal wall than is covered by one pair of NIRS light source and light detector.

In some embodiments, heater controller 16 provides a display, or other readout that displays a graph or other indicia indicating one or more blood-flow-signals for the rectal wall and/or prostate. Heater controller 16 may also display indicia indicating when a criterion for shutting off or reducing the output of heater 12 is almost satisfied or has been satisfied.

The curve shown in FIG. 7 is also representative of the gross behavior of blood flow in the prostate as the prostate receives heat treatment. In some embodiments, heater controller 16 monitors one or more prostate blood-flow signals derived from one or more outputs of NIRS system 22 that are indicative of blood flow in the prostate. In such embodiments, heater controller 16 may be configured to continue operation of heater 12 until the one or more prostate blood-flow signals indicate that the heat from heater 12 has succeeded in damaging tissues of the prostate. For example, heater controller 16 could seek to continue operating heater 12 until one or more prostate blood-flow signals from NIRS system 22 indicates that:
the blood flow in the prostate has peaked and started to decline;
the blood-flow in the prostate is decreasing with time at a rate exceeding a threshold;
the prostate-blood-flow signal is less than or equals a threshold value;
a combination thereof; or
the like.

Where the prostate-blood-flow signal is compared to a threshold, the threshold may be based on one or more of:
a value of the prostate-blood-flow signal prior to or at the outset of treatment;
a value of the prostate-blood-flow signal its peak;
a predetermined value;
a value of the prostate-blood-flow signal at a time when a rate of change of the prostate-blood-flow signal with time reaches a threshold amount; and
the like.

For example, unless a rectal-wall-blood-flow signal satisfies a criterion that causes heater controller 16 to discontinue a treatment, heater controller 16 may be configured to continue treatment (by operating heater 12 continuously or intermittently) until one or more of the following is satisfied:
a predetermined time has elapsed since treatment commenced (the time may be measured, for example, by a timer or timing function built into the controller);
the prostate-blood-flow signal has fallen to a value that is lower than a first threshold;
the prostate-blood-flow signal has fallen to a value that is lower than a fixed or determined percentage of the value that it had at one or more of: a time before, at or just after commencement of treatment; its peak; a time when it had a rate of change more negative than a threshold; or the like;
a predetermined time has passed since a peak of the prostate-blood-flow signal;
etc.

In some embodiments, heater controller 16 comprises a data processor that executes software instructions that cause it to control heater 12 as described herein. The software instructions may be stored in a memory accessible to the data processor. Aspects of the invention may be provided in the form of program products. The program products may comprise any medium which carries a set of computer-readable instructions which, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like. The computer-readable instructions on the program product may optionally be compressed or encrypted.

In such embodiments, thresholds or other parameters that regulate the operation of heater controller 16 and/or functions that represent criteria for determining whether heater 12 should be shut down or have its output reduced may be stored in a memory accessible to the processor.

While a number of exemplary aspects and embodiments have been discussed above, those of skill in the art will recognize certain modifications, permutations, additions and sub-combinations thereof. For example:
instead of detecting blood flow in the prostate by way of NIRS, blood flow in the prostate may be detected by Doppler ultrasound.
It is not mandatory that catheter 30 be hollow or have a lumen capable of carrying fluids in all embodiments. Catheter 30 may be hollow. The term catheter, as used herein, includes elongated flexible structures that can be inserted into a male person's urethra but does not require the presence of a lumen or other hollow passage except as otherwise noted or necessarily implied.

It is therefore intended that the following claims and any claims hereafter introduced are interpreted to include all such modifications, permutations, additions and sub-combinations as are within their true spirit and scope.

1. A system for measuring blood flow in a subject's prostate gland, the system comprising:
a catheter sized and configured such that, when placed within the urinary tract of a male individual, a proximal portion of the catheter resides outside the body of the individual, a middle portion of the catheter resides within a urethra of the individual and a distal portion of the catheter comprising an anchor resides within a urinary bladder of the individual; and,
a blood flow sensor supported by the middle portion of the catheter at a location spaced apart from the anchor.

2. A system according to claim 1 wherein the blood flow sensor comprises at least one near infrared light source and at least one near infrared light detector.

3. A system according to claim 2 wherein the near infrared light source comprises a light-emitting area of a first optical fiber extending along the catheter from the proximal portion to the middle portion and having a light emitter configured to direct light into the optical fiber at a proximal end thereof.

4. A system according to claim 3 wherein the near infrared light detector comprises a light-capturing area of a second optical fiber extending along the catheter from the proximal portion to the middle portion.

5. A system according to claim 4 wherein the first and second optical fibers are coaxial.

6. A system according to claim 5 wherein the first and second optical fibers are slidable relative to one another so as to permit varying a separation between the light-emitting area of the first optical fiber and the light-capturing area of the second optical fiber.

7. A system according to claim 2 wherein the at least one near infrared light detector of the blood flow sensor comprises a plurality of near infrared light detectors spaced apart from one another along the catheter.

8. A system according to claim 7 wherein the at least one near infrared light detector of the blood flow sensor comprises a plurality of near infrared light detectors oriented to detect light incident on the near infrared light detectors from a corresponding plurality of different directions.

9. A system according to claim 2 wherein the at least one near infrared light source and at least one near infrared light detector are arranged to provide a plurality of light-source-detector pairs wherein, in different ones of the light-source-detector pairs corresponding ones of the light sources and light detectors are spaced apart from one another by different distances.

10. A system according to claim 2 wherein the at least one near infrared light source is configured to emit light in a plurality of radial directions relative to the catheter.

11. A system according to claim 10 wherein the at least one near infrared light detector comprises a plurality of near infrared light detectors oriented to detect light incident on the catheter from different radial directions.

12. A system according to claim 2 wherein the catheter comprises a lumen having transparent walls and the at least one near infrared light source and the at least one near infrared light detector are located within the lumen.

13. A system according to claim 2 comprising a near infrared spectroscopy system configured to determine, based at least in part on an output of the near infrared light detector, a value indicative of a concentration of at least one bio compound related to blood flow.

14. A system according to claim 13 wherein the at least one bio compound comprises one or more of oxygenated hemoglobin; non-oxygenated hemoglobin; total hemoglobin; and myoglobin.

15. A system according to claim 2 comprising an opaque barrier between the at least one near infrared light source and at least one near infrared light detector.

16. A system according to claim 15 wherein the opaque barrier has a labyrinth structure and is apertured to permit fluid flow past the opaque barrier.

17. A system according to claim 1 comprising a microwave antenna supported in or on the catheter.

18. A system according to claim 1 comprising a heater for heating the subject's prostate gland and a heater controller connected to control operation of the heater based at least in part on an output of the blood flow sensor.

19. A system according to claim 18 wherein the controller is configured to detect a peak in a blood flow in the subject's prostate based on the output of the blood flow sensor.

20. A system according to claim 19 wherein the controller is configured to determine the end of a predetermined interval after detecting the peak in the blood flow in the subject's prostate.

21. A system according to claim 20 wherein the controller is configured to discontinue operation of the heater at the end of the predetermined time.

22. A system according to claim 19 wherein the controller is configured to discontinue operation of the heater upon detecting the peak.

23. A system according to claim 19 wherein the controller is configured to discontinue operation of the heater upon the output of the blood flow sensor falling to below a threshold value.

24. A system according to claim 18 comprising a rectal probe, the rectal probe comprising a temperature sensor.

25. A system according to claim 18 comprising a rectal probe, the rectal probe comprising a rectal wall blood flow sensor.

26. A system according claim 25 wherein the rectal wall blood flow sensor comprises a near infrared spectroscopy sensor.

27. A system according to claim 26 wherein the near infrared spectroscopy sensor comprises a plurality of pairs of near infrared light sources and corresponding near infrared light detectors.

28. A system according to claim 26 wherein the near infrared spectroscopy sensor comprises a light source and a plurality of different light detectors spaced apart from the light source by different distances.

29. A system according claim 25 wherein the rectal wall blood flow sensor comprises an ultrasound-based blood-flow detector.

30. A system according claim 25 wherein the rectal wall blood flow sensor comprises a skin impedance detector.

31. A system according to claim 25 wherein the rectal probe comprises a temperature sensor.

32. A system according to claim 25 wherein the controller is configured to detect a peak in a signal derived from an output of the rectal wall blood flow sensor.

33. A system according to claim 25 wherein the controller is configured to inhibit operation of the heater if an output of the rectal wall blood flow sensor corresponds to a blood flow that is less than a threshold value.

34. A system according to claim 33 wherein the controller is configured to compute the threshold value based at least in part on a magnitude of a peak in a signal derived from an output of the rectal wall blood flow sensor.

35. A system according to claim 33 wherein the controller is configured to compute the threshold value based at least in part on a magnitude of a signal derived from an output of the rectal wall blood flow sensor at a time before commencing heating.

36. A system according to claim 35 comprising a memory wherein the controller is configured to store a value representing the magnitude of the signal derived from the output of the rectal wall blood flow sensor in the memory.

37. A system according to claim 33 wherein the controller is configured to compute the threshold value based at least in part on a value of the output of the rectal wall blood flow sensor at a time when a rate of change of the value of the output of the rectal wall blood flow sensor exceeds a second threshold.

38. A system according to claim 25 comprising a readout configured to display an indication of a rectal wall blood flow as determined from an output of the rectal wall blood flow sensor.

39. A system according to claim 18 wherein the controller is configured to operate the heater to apply heat to tissues of the subject's prostate during periods in which blood flow as determined by the blood flow sensor is below a threshold value and to not apply heat during periods wherein the blood flow determined by the blood flow sensor exceeds the threshold value.

40. A system according to claim 1 comprising a readout configured to display a blood flow based on an output of the blood flow sensor.

41. A system according to claim 18 wherein the heater comprises a microwave heater.

42. A system according to claim 18 comprising a blood flow control mechanism wherein the system is configured to operate the blood flow control mechanism to reduce a blood flow to the subject's prostate while operating the heater.

43. A catheter useful for measuring blood flow in a subject's prostate gland, the catheter comprising:
an elongated catheter body sized and configured such that, when placed within the urinary tract of a male individual, a proximal portion of the catheter body resides outside the body of the individual, a middle portion of the catheter body resides within a urethra of the individual and a distal portion of the catheter body comprising an anchor resides within a urinary bladder of the individual; and,
a blood flow sensor supported by the middle portion of the catheter body at a location spaced apart from the anchor.

44. Apparatus for applying thermal treatment to a subject's prostate gland, the system comprising:
a rectal probe comprising a rectal wall blood flow sensor;
a tissue heating system;
a control controlling operation of the tissue heating system and connected to receive a rectal wall blood flow signal output by the rectal wall blood flow sensor;
wherein the control is configured to inhibit operation of the tissue heating system upon determining that the rectal wall blood flow signal satisfies a condition.

45. Apparatus according to claim 44 wherein the condition comprises the rectal-wall-blood-flow signal having fallen to a value that is lower than a threshold.

46. Apparatus according to claim 44 wherein the condition comprises the rectal-wall-blood-flow signal is decreasing with time at a rate exceeding a threshold.

47. (canceled)

48. (canceled)
